(19) United States
(12) Patent Application Publication
(10) Pub. No.: US 2024/0197298 A1
(43) Pub. Date: Jun. 20, 2024

(54) ENDOSCOPE IN COMBINATION WITH A LARGE MASS REMOVAL APPARATUS

(71) Applicants: Sharon A. Hibbs, Overland Park, KS (US); Michael McGee, Kansas City, MO (US); Thomas Shireman, Kansas City, MO (US); James M. Walden, JR., Kansas City, MO (US)

(72) Inventors: Sharon A. Hibbs, Overland Park, KS (US); Michael McGee, Kansas City, MO (US); Thomas Shireman, Kansas City, MO (US); James M. Walden, JR., Kansas City, MO (US)

(21) Appl. No.: 18/343,457

(22) Filed: Jun. 28, 2023

Related U.S. Application Data
(60) Provisional application No. 63/476,115, filed on Dec. 19, 2022.

(51) Int. Cl. A61B 10/04; A61B 10/02; A61B 17/32

(52) U.S. Cl. CPC A61B 10/04; A61B 10/0266; A61B 10/0283; A61B 17/320016; A61B 2017/32004

(57) ABSTRACT

An instrument for use in the gastrointestinal tract of a patient comprises a sleeve, an endoscope, a vacuum tube alternatively reciprocal relative to the sleeve and a switching block to control alternative insertions of the endoscope and vacuum tube into the sleeve while directing them into position.

### CROSS-REFERENCE TO RELATED APPLICATIONS

This application claims the benefit of U.S. Provisional Application Ser. No. 63/476, 115, filed Dec. 19, 2022, the disclosure of which is hereby incorporated herein in its entirety by reference.

### BACKGROUND OF THE INVENTION

The invention is to the combination of a relatively large suction or vacuum tube that can easily be used interchangeably with a conventional endoscope, but not at the same time while maintaining the device not in use in a ready position, within a sleeve to provide for the removal of large masses, especially clotted blood, from the stomach or other regions of the body into which the sleeve is inserted.

Doctors often encounter situations within a patient where large amounts of blood have coagulated in clumps, or other large debris is located in the stomach or elsewhere, the size of which makes it hard to remove with the vacuum line accompanying the conventional endoscope and that also make it difficult for the doctor to visualize the area through the endoscope and perform medical activity to correct the cause of the bleeding or to see and correct other issues that are producing a medical problem.

Unfortunately, the cross sectional size to which the esophagus in the throat can expand limits the size of endoscopes. Consequently, when the other features of the endoscope are included, there is only a limited space for a vacuum line. Such vacuum lines cannot handle larger clumps and/or take a comparatively long time to remove blood and debris, especially when there is a lot of it. In particular endoscopes for upper and lower gastrointestinal tracks are frequently equipped with a light source, a water spray, an optical viewing system and a vacuum system. The vacuum system associated with the conventional endoscopes is only suitable for removal of small volumes of liquid or of small particles. The large clumps, especially found with significant bleeding, cannot be easily removed by the comparatively narrow vacuum tube found in conventional endoscopes.

### SUMMARY OF THE INVENTION

An apparatus including a guide block or structure that is designed to easily, interchangeably and not coincidentally receive a conventional endoscope and a vacuum tube in an outer sleeve that fits snugly in the esophagus of a patient. The vacuum tube is sized to fully fill the sleeve that is inserted into the throat of the patient through the esophagus. The sleeve is secured to a bottom side of a switching block that has a first axial passageway through the block to the top thereof. The first passageway is sized and shaped to receive the endoscope. A second passageway asymptotically merges with the first passageway at a junction. In this way either the endoscope or the vacuum tube can be selectively fed through the switching block and sleeve into the stomach while the other device is maintained in a ready position. This allows effective use of either instrument.

The vacuum tube is slidably received to allow relative rotation or up and down relative movement. The lower end of the vacuum tube is extendable past the sleeve. The vacuum tube and endoscope are reciprocal with respect to the sleeve to allow for easy removal or replacement. A stop is provided to limit how far the vacuum tube can advance relative to the sleeve. Different patients have different size esophageal openings, consequently it is foreseen that the apparatus will be constructed in different diameters, especially for children and adults. Preferably, the vacuum tube is sized to be as large as possible and to still be reciprocally received in the sleeve to maximize its capacity. The endoscope can be various sizes but normally will fit snugly but slidably in the sleeve.

Various objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a side elevational view of a switching block with a depending tube for use in accordance with the invention.

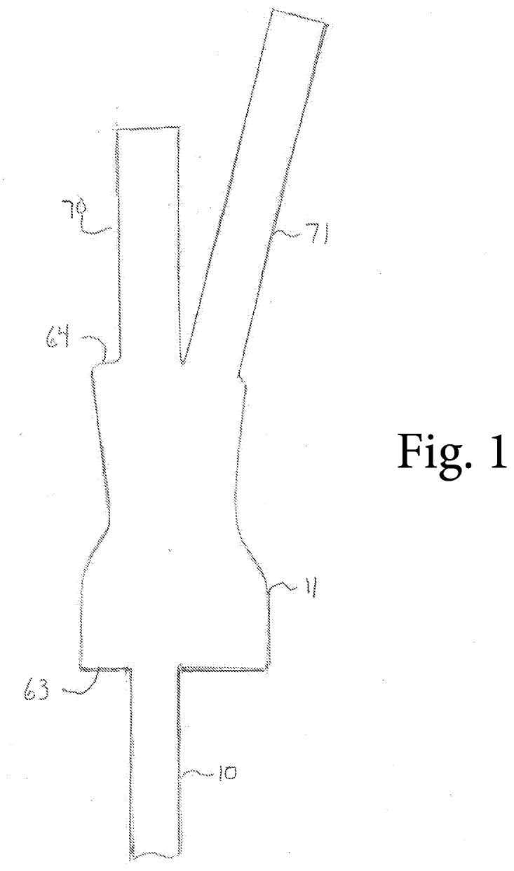

FIG. 2 is a side elevational view of the switching block with portions removed to show detail thereof and in conjunction with an endoscope and suction tube with the endoscope fully inserted and the vacuum tube partially withdrawn.

FIG. 3 is a partially schematic prospective view of the block shown in conjunction with an endoscope and a vacuum tube with the sleeve inserted in a patient and the various equipment to operate the endoscope and vacuum tube.

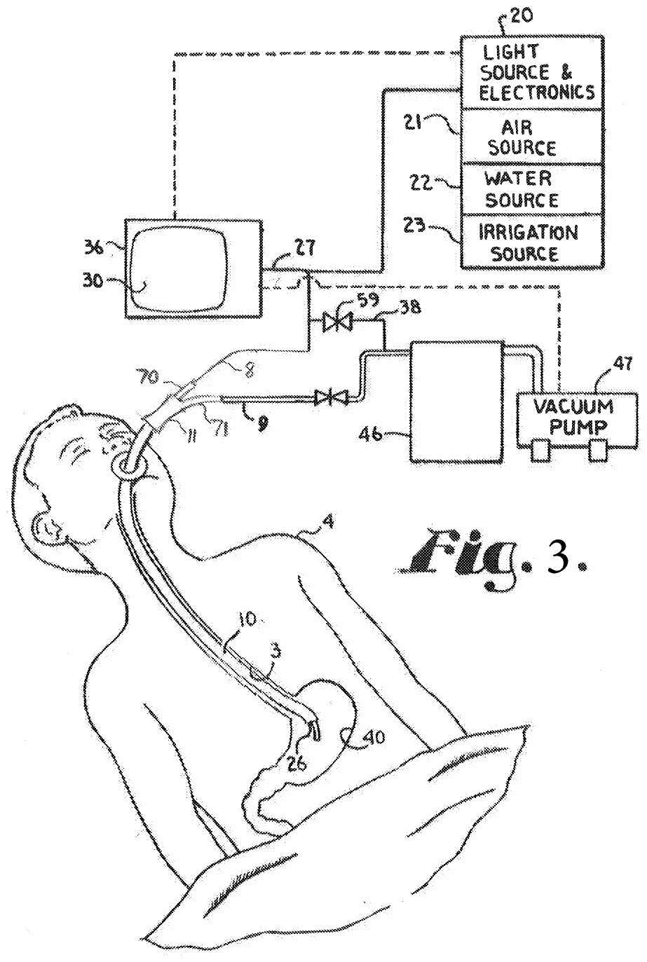

### DETAILED DESCRIPTION OF THE DISCLOSURE

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The reference numeral 1 generally represents an apparatus, assembly or instrument for medical use to conduct medical procedures along the gastrointestinal tract 3 of a patient 4.

The apparatus 1 includes an endoscope 8, a vacuum tube 9, a sleeve 10 and a switching block 11. It is foreseen that the endoscope 8 could be either an upper gastrointestinal tract endoscope or a lower gastrointestinal tract endoscope or any similar scope, but it is preferred that the endoscope 8 be an upper gastrointestinal tract scope, especially having a length of approximately 53 centimeters, because this is the length of the normal or average upper human gastrointestinal tract, although length can be varied with use or person to be used upon.

The endoscope 8 includes an elongate outer sheath 14 encompassing internal channels and electrical conduct 16. Endoscopes vary somewhat in what each include and the size, shape, and layout of the various channels and conducts incorporated therein and it is foreseen that virtually any such endoscope could be used in conjunction with the invention. Typical endoscopes can be seen in thousands of patents, such as U.S. Pat. No. 8,016,753, incorporated herein by reference.

The endoscope 8 illustrated has a body 19 that includes a light, air tube, and multiple water spray tubes (not individually shown), each joined to a light source 20, an air source 21, a water spray source 22, which provides a light spray, and an irrigation source 23 that provides a heavier flow respectively, all at a lower end or tip 26 of the endoscope 8. Such also provide optics 27 through a camera, fiber optics, or the like, that allow a physician conducting the procedure using the endoscopes 8 to view images at the tip 26 of the endoscope 8 to view what is taking place at or near the tip 26 on a screen 30, or the like. The optics are herein included in an elongated shaft 33 that can be reciprocated within the body 19 that receives the shaft 33. The various sources for light 20, air 21, water 22 and 23 are all centrally controlled through a console 36 that has conventional keyboard control, or the like, and the monitor 30.

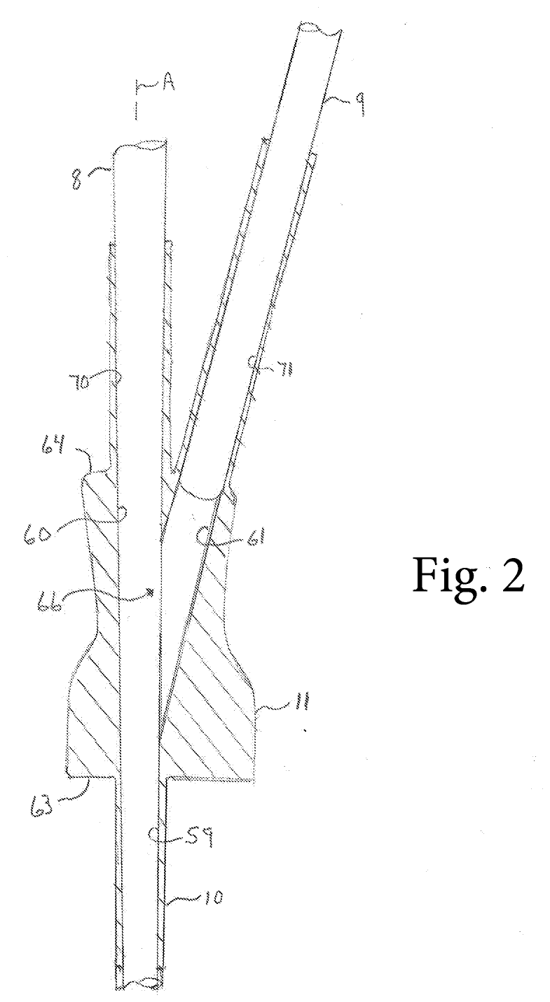

The endoscope 8 also typically incorporates a vacuum lumen 38 that is relatively narrow and typically about 4 to 6 french in size. The lumen 38 with an opening at or near the tip 26 and that is designed to remove water from sources 22 and 23, as well as other light fluids and possibly small solids from the gastrointestinal area, especially the stomach 40. The vacuum lumen 38 discharges into a collection chamber 46 which in turn is fluidly connected to a vacuum pump 47 which provides the vacuum to the lumen 38. The pump 47 is controlled by the operator through the console 36. The endoscope shaft 33 can be retracted or withdrawn into the sleeve 10 or, as shown in FIG. 2, can be extended from the sleeve 10. Typically, the overall endoscope 8 is sized in the range of 4 to 10 french in cross section.

The vacuum tube 9 is elongate and extends along the sleeve 10 so when inserted therein the vacuum tube 9 and the endoscope 8 are useable interchangeably in the sleeve 10, but portions thereof may remain in the switching block 11 above a juncture 66 when not in use. Both the tube 9 and lower part of the endoscope 8 are generally encircled by the sleeve 10, but not at the same time. The tube 9 is preferably about 53 centimeters in length which is the common length of the human adult upper gastrointestinal tract, but it is foreseen that it can be shorter or longer as patient size or circumstances warrant. The vacuum tube 9 is sized in cross section to approach the inner size of the sleeve 10, that is within the sleeve lumen 59, while still being receivable through the esophageal passage to allow for the largest interior cross section of the vacuum tube 9. Preferably, the vacuum tube 9 cross sectional size is at least approximately 46 french when used in adults. The total cross sectional size of the instrument 1 is normally maintained under 50 french to fit in the esophagus opening of most adult patients. This, in turn, allows removal of large debris, such as food particles, ingested nonfood items, and congealed blood. Removal of a large accumulation of congealed blood is the major purpose of the tube 9.

Preferably, the sleeve to be as large as can be normally received through the esophagus and vacuum tube to be cross-sectionally received in the sleeves while still be reciprocally movable in the sleeve to allow the cross-section of the vacuum tube to be maximized.

Located at a lower end of the vacuum tube 8 in a soft pliable sleeve 55 that is slightly tapered toward center and which acts as a barrier to prevent the vacuum tube 9 from hitting the wall of the stomach 40 or other internal structure and doing damage to the structure. The sleeve 55 is preferably an angular structure with a central axis A and an angular wall with of about 1 millimeter or larger where strength is required. The sleeve 55 is preferably constructed of a soft pliable silicone.

An upper end 56 of the tube 9 is joined to a vacuum line 57, which in turn is fluidly joined to the collection chamber 46. In this manner, when the vacuum pump 47 is on, the tube 9 can draw large pieces or clumps of debris through the sleeve 55 and eventually into the collection chamber 46. Flow through the vacuum tube 9 into the collection chamber 46 is preferably controlled by a valve 58; however, it is foreseen that a nurse or other medical practioner could simply disconnect the tube 10. A valve 59 also controls the vacuum line of the endoscope 8.

As can be appreciated, the vacuum tube 9 allows removal of large debris, especially large clots of blood which cannot be removed by conventional endoscopes. This allows a physician to see and treat a source of a medical problem quicker than conventional procedures.

The switching block 11 allows the endoscope 8 to be effectively and efficiently used in conjunction with each other. The sleeve 10 maintains the throat of the patient open while interchange occurs and first and second passageways 60 and 61 of the block 11 provide quick and easy access as well as guiding to the sleeve 10.

The block 12 has an outer shape that can be selected from any shape easily handled by the doctors preforming the procedure. The block 12 has a bottom surface 63 that extends toward the stomach 40 of the patient 4 and a top surface 64. The top and bottom surfaces 63 and 64 are intersected by the first passageway 60. The second passageway 61 also intersects the top surface 64 at a distance from the first passageway 60. The first and second passageways 60 and 61 openly meet at a juncture 66. In this manner the endoscope 8 may be selectively inserted through the passageway 60 into the sleeve 11 and then the stomach 40 or removed and alternatively the vacuum tube 9 may be inserted through the passageway 61 asymptotically in a manner like a car enters a freeway from a cloverleaf turn and then guided forward to the sleeve 11 and stomach 40. In this way the endoscope can perform various endoscopic functions and the vacuum tube 9 can remove large debris from the stomach 40 both quickly and efficiently.

The upper ends of passageways 60 and 61 are extended above the switching block 11 by elongate tubular extensions 70 and 71 which are the same internal cross-sectional size as the passageways 60 and 61 respectively and the sleeve 10. Preferably, the inner diameter of the passageways and sleeve 10 are equal.

The vacuum tube 9 is numbered from bottom incrementally in centimeters to allow physicians to determine the proximate depth of the bottom of the vacuum tube 9 in the stomach.

It is to be understood that while certain forms of the present invention have been illustrated and described herein, it is not to be limited to the specific forms or arrangement of parts described and shown.

1. An apparatus for performing medical procedures in a patient comprising:
a) an elongate endoscope having an optical viewing system;
b) an elongate vacuum tube, the vacuum tube having a lower opening to allow debris to be drawn into the tube when under vacuum, and a vacuum system joined to the vacuum tube for drawing a vacuum therein;
c) a sleeve alternatively surrounding both the endoscope and the vacuum tube; and
d) a switching block with a pass through first passageway; an upper end of the first passageway receiving the endoscope and a second passageway joining with the first passageway at a junction whereby the endoscope is selective placeable in and withdrawable from both the sleeve in a patient's stomach and the vacuum tube is interchangeably positionable in the sleeve and stomach when the endoscope is withdrawn sufficiently so as to clear the junction.

2. The apparatus according to claim 1 wherein the sleeve is cross-sectionally sized such that both the vacuum tube and endoscope cannot be simultaneously received in the sleeve to allow maximization of the cross-sectional size of the vacuum tube.

3. The apparatus according to claim 1 wherein:
a) the first and second passageways each include upper portions above the juncture such that the endoscope and suction tube can both remain at least partially in the respective passageways thereof when the opposite of the endoscope and vacuum tube are received in the sleeve.

4. A switching block for use in suctioning contents of a patient's stomach comprising:
a) a body adapted to be held by a physician;
b) a first passageway for receiving an endoscope within the body;
c) a second passageway for receiving a suction tube in the body; and joining at a junction.
